SEQUENCE LISTING (35 sequences)

SEQ ID NO: 1 - DNA, 6 bp, Homo sapiens
ttaggg

SEQ ID NO: 2 - DNA, 24 bp, Homo sapiens
tggatgagaatggcaaaattagct

SEQ ID NO: 3 - DNA, 19 bp, Homo sapiens
caccccagcaccacattca

SEQ ID NO: 4 - DNA, 20 bp, Homo sapiens
cccacactgcagcagatcag

SEQ ID NO: 5 - DNA, 22 bp, Homo sapiens
cacactcggaccacatccttct

SEQ ID NO: 6 - DNA, 23 bp, Homo sapiens
cctccatggatctgcttattcag

SEQ ID NO: 7 - DNA, 23 bp, Homo sapiens
tgcgacactattctctgcagaag

SEQ ID NO: 8 - DNA, 23 bp, Homo sapiens
gaaagcatctcctcattcatggt

SEQ ID NO: 9 - DNA, 20 bp, Homo sapiens
gctctcaacgaacgctttcc

SEQ ID NO: 10 - DNA, 21 bp, Homo sapiens
gagaaccccaagatgcacaac

SEQ ID NO: 11 - DNA, 19 bp, Homo sapiens
cgcttagcctcgtcgatga

SEQ ID NO: 12 - DNA, 20 bp, Homo sapiens
ggtcgttcatgcccaacttg

SEQ ID NO: 13 - DNA, 21 bp, Homo sapiens
ccggtggatgtcatcaaagtc

SEQ ID NO: 14 - DNA, 17 bp, Homo sapiens
gcaagggccccatcaac

SEQ ID NO: 15 - DNA, 22 bp, Homo sapiens
ggtctgtcccattgagcttctc

SEQ ID NO: 16 - DNA, 21 bp, Homo sapiens
ccacccaagttcgacaaaatc

SEQ ID NO: 17 - DNA, 22 bp, Homo sapiens
cgtagcgatccttgagcttcta

SEQ ID NO: 18 - DNA, 22 bp, Homo sapiens
tgagaaagaagggccttaatgg

SEQ ID NO: 19 - DNA, 21 bp, Homo sapiens
cagggctgtgacctacggaat

SEQ ID NO: 20 - DNA, 18 bp, Homo sapiens
ccccccagtggatgtctt

SEQ ID NO: 21 - DNA, 18 bp, Homo sapiens
ccggaacccttccatgtg

SEQ ID NO: 22 - DNA, 21 bp, Homo sapiens
cgtctccacacatcagcacaa

SEQ ID NO: 23 - DNA, 22 bp, Homo sapiens
tcttggcagcaggatagtcctt

SEQ ID NO: 24 - DNA, 19 bp, Homo sapiens
cgccacccacacttgtgat

SEQ ID NO: 25 - DNA, 22 bp, Homo sapiens
gtgccttgagatgggaactctt

SEQ ID NO: 26 - DNA, 24 bp, Homo sapiens
caagttctgggaagtcatcagtga

SEQ ID NO: 27 - DNA, 19 bp, Homo sapiens
ccgagtcgcccacgtagtt

SEQ ID NO: 28 - DNA, 24 bp, Homo sapiens
tcctgctgtagatgcagagatcag

SEQ ID NO: 29 - DNA, 18 bp, Homo sapiens
aggacgctggcaggaatg

SEQ ID NO: 30 - DNA, 23 bp, Homo sapiens
gagatccgcacgcagcagtatga

SEQ ID NO: 31 - DNA, 22 bp, Homo sapiens
acttggagcggtaccactcttc

SEQ ID NO: 32 - DNA, 20 bp, Homo sapiens
ttcctcccaaggcacagaga

SEQ ID NO: 33 - DNA, 18 bp, Homo sapiens
ccctgtcaccgccaaaga

SEQ ID NO: 34 - DNA, 23 bp, Homo sapiens
tcagtggtgtggaatgatgattc

SEQ ID NO: 35 - DNA, 16 bp, Homo sapiens
ccggcgtcgtcgatgt

(19) United States
(12) Patent Application Publication
(10) Pub. No.: US 2011/0293572 A1
(43) Pub. Date: Dec. 1, 2011

(54) INCREASING THE PLASTICITY OF STEM CELLS

(76) Inventors: PAUL GENEVER, FANGFOSS (GB); ELLEN BRAY, YORK (GB)

(21) Appl. No.: 12/935,108

(22) Filed: Sep. 28, 2010

(30) Foreign Application Priority Data
Mar. 28, 2008 (GB) 0805670.7
Mar. 27, 2009 (GB) PCT/GB09/00833

(51) Int. Cl. A61K 35/12; C12N 5/0775; C12N 5/078; C12N 5/071

(52) U.S. Cl. 424/93.7; 435/375; 435/325

(57) ABSTRACT

The invention relates to methods of culturing non-embryonic cells to increase their plasticity and their potential to differentiate into multi-lineage cell types.

This application is a U.S. National Stage Application, submitted under 35 U.S.C. 371, claiming priority to PCT International Patent Application PCT/GB09/000,833 filed on Mar. 27, 2009, which claims priority to UK provisional application no. 0805670.7, filed on 28 Mar. 2008, the disclosures of which are incorporated by reference in their entireties.

### FIELD OF THE INVENTION

The invention relates to methods of culturing non-embryonic cells to increase their plasticity and their potential to differentiate into multi-lineage cell types.

### BACKGROUND TO THE INVENTION

Stem cell therapies are expected to provide treatments for a large range of pathologies and disorders. Stem cells are capable of undergoing a self-renewing cell division or differentiating into multi-lineage cell types depending on the biological cues that are present in their particular niches.

Research has mainly been conducted on embryonic stem cells (ESCs), obtained from the blastocyst or embryonic germ, or mesenchymal stem cells (MSCs) derived from adult tissue sources (bone marrow). ESCs are believed to hold the greatest multi-potency, with MSC's postulated as having a lower self-renewal capacity.

There is a need for a new stem cell source which exhibits a greater plasticity in terms of differentiation and proliferation than adult MSCs and which is free from the ethical considerations associated with ESC sources and the complications involved with generating patient specific cells.

In an attempt to solve this problem, Takahashi, K. et al (2007) reprogrammed differentiated human somatic cells into a pluripotent state to allow the creation of patient- and disease-specific stem cells. They transduced human dermal fibroblasts with the transcription factors Oct3/4, Sox2, Klf4, and c-Myc, genes primarily expressed in ESCs. These so called induced pluripotent stem (iPS) cells were similar to human ESCs in morphology, proliferation, surface antigens, gene expression, epigenetic status of pluripotent cell-specific genes and telomerase activity.

Likewise, Yu, J. et al (2007) induced the conversion of human somatic cells to pluripotent stem cells having an embryonic stem cell phenotype by transducing the somatic cells with OCT4, SOX2, NANOG, and LIN28.

The techniques of Takahashi, K. et al and Yu, J. at al both require complicated genetic manipulation of cells. This substantially increases the culture time required between removal of the cells from the subject and reimplantation. Furthermore, as a result of the large number of retroviral integration sites present in the generated IPS cells there may be an increased chance of tumourigenesis.

By removing two-dimensional growth constraints associated with monolayer techniques it is possible to induce the formation of three-dimensional cellular cultures referred to as ‘spheroids’. By culturing non-embryonic stem cells as spheroids at a defined seeding density for a defined culture period using standard culture reagents, we have demonstrated these cells can be re-programmed to express genes characteristic of an embryonic cell type. These cells exhibit a greater plasticity than the non-embryonic stem cells and have the capability to differentiate into multi-lineage cell types.

### SUMMARY OF THE INVENTION

According to a first aspect of the invention there is provided an in vitro method of culturing non-embryonic cells such that the cells revert to a cell type expressing at least one gene expressed by an embryonic or embryonic-like cell, the method comprises the steps of;
i) providing non-embryonic cells,
ii) culturing the cells as a cellular aggregate for a period of between about 4 and 8 days at an initial cell density per aggregate of between about 3×10⁴ and 12×10⁴ cells.

The cells obtained from this method exhibit pluripotent characteristics and as such this method may be considered as a method of enhancing the pluripotency of a cell.

In particular the cells revert to a cell type expressing at least one gene expressed by an embryonic cell or embryonic-like cell.

In particular the cells revert to a cell type expressing at least one gene expressed by an embryonic stem cell or embryonic-like stem cell.

In embodiments of the invention the non-embryonic cells are multipotent cell, for example, non-embryonic stem cells.

The term “non-embryonic stem cell” encompasses any stem cell not derived from an embryo and can be derived from any fetal, neo-natal or adult tissue.

An adult stem cell also referred to as a somatic stem cell is an undifferentiated cell found among differentiated cells in a tissue or organ.

Suitable sources of non-embryonic stem cells include, but are not limit to: bone marrow, bone marrow aspirates, adipose tissue, Wharton's Jelly and umbilical cord blood.

Non-embryonic stem cells include mesenchymal (also referred to as marrow stromal cells) or haematopoietic stem cells.

Mesenchymal adult stem cells can form a variety of cell types including fat cells, cartilage, bone, tendon and ligaments, muscles cells, skin cells and nerve cells. In embodiment's of the invention the non-embryonic cells are non-embryonic mesenchymal stem cells.

Haematopoietic adult stem cells are found mainly in the bone marrow and they differentiate into the various types of blood cell. In embodiments of the invention the non-embryonic cells are non-embryonic haematopoietic stem cells.

In embodiments of the invention the non-embryonic cells are progenitor cells. Progenitor cells are herein defined as immature or undifferentiated cells, typically found in post-natal animals. In comparison to “true” stem cells which are characterised by an unlimited self-renewal capability and pluripotency, progenitor cells have a more restricted self-renewal capability and are unipotent or multipotent.

In embodiments of the invention the non-embryonic cells are partially or terminally differentiated cells, for example, fibroblasts, or chondrocytes.

The cells are not cultured in mono-layer but are cultured as a 3D culture.

In embodiments of the invention the cells are cultured as spheroids. As used herein, the term spheroid refers to a three-dimensional structure, normally spherical in shape, which does not occur in nature and which consists of a re-aggregate of cells.

In embodiments of the invention the cells are cultured in standard culture media in the presence of an agent which promotes cell aggregation. Examples of suitable agents include methyl cellulose, fibrin or thrombin.

In alternative embodiments of the invention the cells are seeded into a 3D construct which permits the cells to be cultured in vitro as an aggregate. Such a construct could be made from a gel.

In embodiments of the invention the 3D construct is substantially spherical.

In embodiments of the invention the cells are cultured for a period of about 4, 4.25, 4.5, 4.75, 5, 5.25, 5.5, 5.75, 6, 6.25. 6.5, 6.75, 7, 7.25, 7.5, 7.75 or 8 days or alternatively for a time period in between thereof.

In embodiments of the invention the initial cell density per aggregate is between about 3×10⁴ and 9×10⁴ cells, or about 3×10⁴ and 7×10⁴ cells, or about 4×10⁴ and 8×10⁴ cells, or about 4×10⁴ and 8×10⁴ cells, or about 5×10⁴ and 7×10⁴ cells.

In embodiments of the invention the initial cell density of the aggregate is between about 5×10⁴ and 7×10⁴.

In embodiments of the invention the initial cell density of the aggregate is about 5×10⁴ and 7×10⁴ and the cells are cultured for about 5 days.

In a specific embodiment of the invention the cell density per aggregate is about 6×10⁴ cells.

In a further specific embodiment of the invention the cells are cultured at a density of about 6×10⁴ cells per aggregate and cultured for a period of between about 4 and 8 days.

In a further specific embodiment of the invention the cells are cultured at a density of about 6×10⁴ cells per aggregate and cultured for a period of about 5 days.

Following culture the cells revert to a cell type expressing at least one gene which is characteristic of an embryonic or embryonic-like phenotype and can be used as a marker of this phenotype. Examples of such embryonic genes include, but are not limited to:

Octamer-4 (Oct-4) is a homeodomain transcription factor of the POU family. This protein is critically involved in the self-renewal of undifferentiated ESCs and is expressed in developing embryos throughout the pre-implantation period.

Nanog is a transcription factor expressed in (ESCs) and is thought to be a key factor in maintaining pluripotency by acting on concert with Oct4 and Sox2.

The Rex-1 (Zfp-42) gene, which encodes an acidic zinc finger protein, is expressed at high levels ESCs. Rex1 is thought to be positively regulated by oct4 and plays a role in the self renewal of undifferentiated stem cells.

SRY (sex determining region Y)-box 2, also known as SOX2, is a transcription factor that is essential to maintain self-renewal of undifferentiated embryonic stem cells.

Telomerase is an enzyme that adds specific DNA sequence repeats (“TTAGGG” in all vertebrates) to the 3′ (“three prime”) end of DNA strands in the telomere regions, which are found at the ends of eukaryotic chromosomes. The telomeres contain condensed DNA material, giving stability to the chromosomes. The enzyme is a reverse transcriptase that carries its own RNA molecule, which is used as a template when it elongates telomeres, which are shortened after each replication cycle. The protein composition of human telomerase consists of two molecules each of human Telomerase Reverse Transcriptase (hTERT), Telomerase RNA (hTR or hTERC) and dyskerin. Embryonic stem cells express telomerase, which allows them to divide repeatedly and form the individual. In adults, telomerase is expressed in cells that need to divide regularly (e.g., in the immune system), although most somatic cells do not express it.

Other markers characteristic of an embryonic or embryonic-like phenotype include; c-myc, KLF-4 and Lin28.

The expression of these genes by the cells in the aggregate can be determined, for example by PCR techniques using the primers listed in Table 1 or 2.

According to a fourth aspect of the invention there is provided a cell aggregate comprising non-embryonic cells which have reverted to a cell type expressing at least one gene expressed by an embryonic cell or embryonic-like cell obtainable by culture of the non-embryonic stem cells according to the first, second or third aspects of the invention.

According to a fifth aspect of the invention there is provided a cell derived from a cell aggregate comprising non-embryonic cells according to the fourth aspect of the invention.

According to a sixth aspect of the invention there are provided various methods of utilizing the cell aggregates and/or cells derived from said aggregates for therapeutic purposes. These cells exhibit a greater plasticity than the original (pre-cultured) non-embryonic cells.

The cells derived by the culture method of the invention have the potential to differentiate into tissues of the endoderm, mesoderm or ectoderm (incl. neural crest) germ layers.

In embodiments of the invention the cells are capable of differentiation into cells of the haematopoietic lineage and/or mesenchymal lineage.

In embodiments of the invention the cells are exposed to an agent that directs them towards and along a specific lineage.

The cells can be exposed to this agent either prior to and/or during and/or following their delivery to a subject. An example of an agent that can direct the cells towards the osteogenic lineage is bone morphogenetic protein-2 (BMP-2). An example of an agent that can direct the cells towards the chrondrogenic lineage is TGFβ.

The subject can be a human or a non-human animal.

According to a further aspect of the invention there is provided a composition comprising a cell aggregate and/or a cell according to the fourth or third aspects of the invention.

Although the cell aggregates and/or cells of the invention can be administered alone, in preferred embodiments of the invention the cell aggregates and/or cells are utilized in the form of pharmaceutical compositions. Such compositions comprise a therapeutically effective amount of the cell aggregate and/or cells, and a pharmaceutically acceptable carrier or excipient.

Suitable carrier and diluents are those that are biologically and physiologically compatible with the recipient, such as buffered saline solution. Other excipients include water, isotonic common salt solutions, alchols, polyols, glycerine and vegetable oils or combinations thereof. The composition for administration must be formulated, produced and stored according to standard methods complying with proper sterility and stability.

The cell aggregates and/or cells can be administered by a route which is suitable for the particular tissue to be treated. The cells can be administered systemically, i.e, parenterally by for example intravenous, subcutaneous or intramuscular injection.

Alternatively the cells can be delivered locally at the required site in a suitable vehicle or carrier, for example seeded onto a porous scaffold such as a felt or gauze, or administered in a gel, such as a hydrogel or hyaluronic acid.

According to a further aspect of the invention there is provided a composition comprising a cell aggregate and/or a cell of the present invention for use as a medicament.

According to a further aspect of the invention there is provided the use of a cell aggregate and/or a cell of the present invention in the manufacture of a medicament for the treatment of a pathology in which the administration of a cell having an embryonic or embryonic-like phenotype would be therapeutically beneficial.

In embodiments of the invention the pathology relates to tissue derived from the endoderm, mesoderm or ectoderm (incl. neural crest) germ layers.

In particular embodiments of the invention the pathology relates to tissue derived from the mesoderm. An example of such tissue is connective tissue which can be classified as areolar (loose) connective, dense connective, elastic, reticular, and adipose. Specific examples of connective tissue include: bone, cartilage, tendon, ligament, muscle, meniscus, fascia or disc.

Examples of pathologies of connective tissue that are envisaged to be beneficially treated with a cell of the invention, include osteoarthritis and spinal disc degeneration.

According to a further aspect of the invention there is provided a method for repairing connective tissue damage. The method of administering the cell aggregates and/or a cell of the invention to an area of connective tissue damage under conditions suitable for differentiating the cells into the type of connective tissue necessary for repair.

Examples of connective tissue include, but are not limited to, bone, cartilage, tendon, ligament, muscle, meniscus, fascia or disc.

The methods and materials disclosed herein are suitable for use in orthopaedic, dental, oral, maxillofacial, periodontal and other surgical procedures.

According to a further aspect of the invention the aggregates can be used as an in vitro model allowing investigation of the factors important in the maintenance/preservation of the embryonic stem cell phenotype.

According to a further aspect of the invention the aggregates can be used as an in vitro model for characterising embryonic or embryonic-like cells. For example the aggregates can also be used as a model to identify novel markers expressed by non-embryonic cells having an immature phenotype. Such markers can be used to identify and isolate cells that have the potential for multi-lineage differentiation.

According to a further aspect of the invention there is provided a method, cell aggregate, cell, composition or use as substantially herein defined with reference to the accompanying Examples and Figures.

### BRIEF DESCRIPTION OF THE DRAWINGS

FIG. 1: C3H10t1/2 cells that have been formed into spheroids according to the method outlined within Example 1. Images shown have been cultured for 5 days. Images are representative of spheroids containing increasing cell numbers, and have been captured using a light microscope.

FIG. 2: Real-time PCR was performed on RNA isolated from cell spheroids containing human MSCs to detect the expression of Oct4 mRNA. Different numbers of cells per spheroid are shown (x-axis), as well as different culture times (different shaded bars). All numbers are relative to a Oct4 mRNA expression form a population of human MSCs grown in monolayer.

FIG. 3: Real-time PCR was performed on RNA isolated from cell spheroids containing human MSCs to detect the expression of Nanog mRNA. Different numbers of cells per spheroid are shown (x-axis), as well as different culture times (different shaded bars). All numbers are relative to a Nanog mRNA expression form a population of human MSCs grown in monolayer.

FIG. 4. Real-time PCR was performed on RNA isolated from cell spheroids containing human MSCs to detect the expression of Rex-1 mRNA. Different numbers of cells per spheroid are shown (x-axis), as well as different culture times (different shaded bars). All numbers are relative to a Rex-1 mRNA expression form a population of human MSCs grown in monolayer.

FIG. 5. Real-time PCR was performed on RNA isolated from cell spheroids containing human MSCs to detect the expression of Sox-2 mRNA. Different numbers of cells per spheroid are shown (x-axis), as well as different culture times (different shaded bars). All numbers are relative to a Rex-1 mRNA expression form a population of human MSCs grown in monolayer.

FIG. 6: Up-regulation of Oct-4, Nanog, SOX2 and Rex1 in human dermal fibroblasts.

FIG. 7: MSCs cultured in 3D have multi-lineage potential.

FIG. 8: Expression of embryonic genes in spheroids with an initial seeding density of 60,000 cells per spheroid.

FIG. 9: The potential of cultured MSCs to differentiate into cell types from other germ layers, for example cardiomyocytes (FIG. 9A) and neuronal cells (FIG. 9B-D).

FIG. 10: Proliferation rate of cells within the 3D cellular model.

FIG. 11: Are the cells senescent or quiescent?

FIG. 12: Organisation and morphology of MSCs in 3D cellular model.

FIG. 13: Oxygen consumption of MSCs in 3D cellular model.

FIGS. 14A and 14B: In vivo study

### DETAILED DESCRIPTION OF THE DRAWINGS

### Example 1

### Spheroid Production Method Development and Characterisation

### Materials and Methods

A mouse osteogenic cell line (C3H10t1/2) was used for method development. Cells were trypsinised and seeded at a specific cellular density into non adherent 96 well U shaped plates. Cells were resuspended at a density of 3×10⁴, 6×10⁴, 1.2×10⁵, and 2.4×10⁵ in 200 μl of Dulbecco's modified Eagles medium containing 100 U/ml penicillin and 100 μg/ml streptomycin, 15% FBS, and 0.25% methyl cellulose. Spheroids were incubated at 37° C. in 5% CO₂ in 95% air with 90% humidity. Spheroids were cultured for 1-7 days and images captured using a light microscope.

### Results

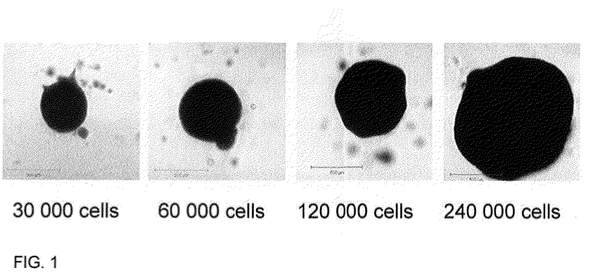

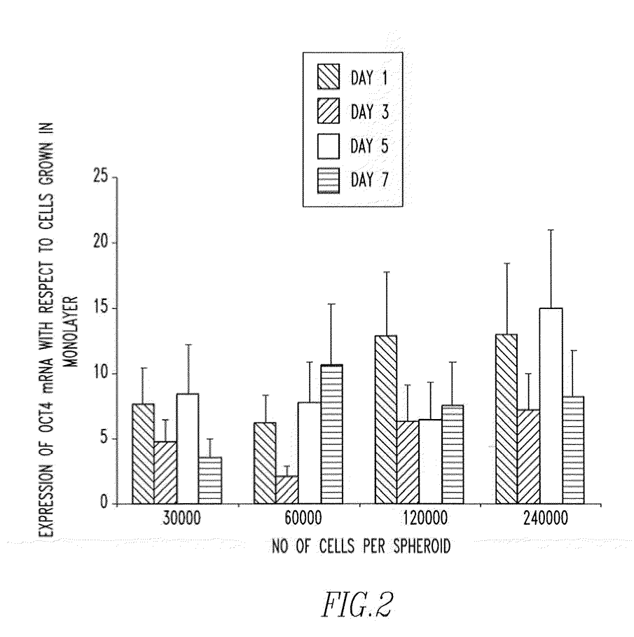

FIG. 1 represents images of spheroids produced using this method, with increasing numbers of cells/spheroid shown.

The method outlined above can be used to produce regular shaped and sized spheroids and as such is a reproducible and reliable model.

### Example 2

### Immature Non-Embryonic Cells Derived from Human MSCs

### Materials and Methods

### i) Cell Culture Medium

Dulbecco's modified Eagles medium containing 100 U/ml penicillin and 100 μg/ml streptomycin, 15% FBS, and 0.25% methyl cellulose. Cells were incubated at 37° C. in 5% CO₂ in 95% air with 90% humidity.

ii) Isolation of Mesenchymal Stem Cells from Femoral Heads

Femoral heads from routine hip replacements were obtained. The trabecular bone was removed from the centre of the femoral head and transferred Dulbecco's modified Eagles medium (DMEM) containing 100 U/ml penicillin and 100 μg/ml streptomycin. The trabecular bone was minced with scissors, fragments allowed to settle and the media transferred to another tube. This was repeated another two times and the bone fragments vortexed before transferring the media.

This cell suspension was centrifuged at 500 g for 5 minutes and the pellet resuspended in 16 ml of DMEM. This suspension was then passed through a 70 μm cell sieve, to remove large debris before being layered over 12 ml of Ficoll-Paque Plus (Amersham Biosciences) and centrifuged at 350 g for 30 minutes. The mesenchymal stem cells were harvested, washed twice in 10 ml phosphate buffered saline (PBS0/0.2% bovine serum albumin (BSA)/5 mM ethylenediaminetetraacetic acid (EDTA), resuspended in DMEM plus 15% batch tested foetal bovine serum FBS (batch tested B) and seeded into a 75 cm² flask. Cells were left to adhere, media changes were carried out every 3-4 days.

iii) Spheroid Production

A proportion of the adherent human bone marrow derived MSCs were trypsinised and seeded at 1000 cells/cm² into non-adherent 96 well U shaped plates for culture in monolayer.

The remaining MSCs were resuspended at a density of 3×10⁴, 6×10⁴, 1.2×10⁵, and 2.4×10⁵ in 200 μl of Dulbecco's Modified Eagles medium containing 100 U/ml penicillin and 100 μg/ml streptomycin, 15% FBS (batch tested for stem cell maintenance and osteogenic capacity), and 0.25% methyl cellulose and cultured in the non-adherent 96 well U shaped plates.

### iv) RNA Extraction

The monolayer and spheroid cultured MSCs were washed in PBS and

RNA extracted in 1 ml Trizol (Gibco, UK) and left for 5 minutes at room temperature. Spheroid MSCs were broken up by passing through a series of needles, (16G, 19G and 21G) during the incubation in trizol.

204 μl of chloroform was added to the Trizol, incubated at RT for 5 minutes prior to being centrifuged for 20 minutes at 12,000 g at 4° C. The aqueous layer was then carefully removed and 500 μl of isopropanol added. The resulting solution was incubated for 30 minutes at 4° C., and centrifuged for 15 minutes at 12,000 g at 4° C. The isopropanol was then removed, and the resulting pellet washed in 75% ethanol, air dried and resuspended in 12 μl DNase and RNase free water before DNase treatment using the DNA-free kit from Ambion.

v) cDNA Synthesis

1 μg RNA samples in a volume of 10 μl plus 1 μl oligo dTs and 1 μl 10 μM dNTPs were incubated for 5 minutes at 65° C. then transferred to ice. 7 μl master mix containing 4 μl of 5× reaction buffer, 2 μl 0.1M DTT and 1 μl DNAse and RNAse free water were added and incubated at 42° C. for 2 minutes. 1 μl of superscript II, or in the case of no RT controls, DNAse and RNAse free water were added and the solution incubated at 42° C. for 50 minutes then 70° C. for 15 minutes. The cDNA was then diluted 1:5 in DNase and RNase-free water.

### vi) Real-Time PCR Primers

Real-time PCR primers for the embryonic markers Oct4, Nanog and Rex1 and the housekeeping gene ribosomal protein subunit 27A (RPS27A) were designed for the SYBR green system using the Applied Biosystems Primer Express software and purchased from Sigma-Genosys. The primer sequences are detailed in Table 1. All real-time PCR reactions were carried out using an ABI Prism 7000 Sequence Detection System (Applied Biosystems).

TABLE 1
Details of the primer sequences for
the qRT-PCR reactions

| Primer Name | Primer Sequences | |
|---|---|---|
| RPS27A | Forward: | TGGATGAGAATGGCAAAATTAGCT |
| | Reverse: | CACCCCAGCACCACATTCA |
| Oct4 | Forward: | CCCACACTGCAGCAGATCAG |
| | Reverse: | CACACTCGGACCACATCCTTCT |
| Nanog | Forward: | CCTCCATGGATCTGCTTATTCAG |
| | Reverse: | TGCGACACTATTCTCTGCAGAAG |
| Rex1 | Forward: | GAAAGCATCTCCTCATTCATGGT |
| | Reverse: | GCTCTCAACGAACGCTTTCC |
| Sox-2 | Forward: | GAGAACCCCAAGATGCACAAC |
| | Reverse: | CGCTTAGCCTCGTCGATGA |

vii) Real Time PCR

The relative expression levels of the embryonic genes Oct4, Nanog, Rex1 and Sox-2 were determined in spheroids with different seeding densities (30, 60 120 and 240 thousand MSCs/spheroid) over seven days in culture and compared to MSCs cultured in monolayer at 1000 cells/cm². cDNA synthesised from 1 μg of RNA was diluted 1:5 for the reactions using the embryonic primers and 1:50 for those using the Housekeeping primers. Reactions were carried out in triplicate in 96-well plates with each well containing 5 μl cDNA, 12.5 μl 2×SYBR Green master mix (Applied Biosystems), 2 μl primer pair mix (containing 10 μM each of forward and reverse primers) and 5.5 μl H₂O, No RT and water controls were also included. Thermal cycling was carried out at 95° C. for 10 minutes, 40 cycles of 95° C. for 15 seconds and 50° C. for 1 minute. Data were analysed using the ABI 7000 System software (Applied Biosystems). The Ct values were normalised against the housekeeping Ct values to obtain the ΔCt values. These values where then normalised to the monolayer Ct values in order to obtain the ΔΔCt values. Two to the power of these values gave 2^{-ΔΔCt}, which was then averaged to give the fold change of each of the sample groups. Using the following equation =IF(A>=1,(A),(−1/A)) the actual fold change was then calculated and plotted (Livak, 2001)
A=average fold change previously calculated

### Results

i) Oct-4 mRNA Expression in Human MSC Cell Spheroids

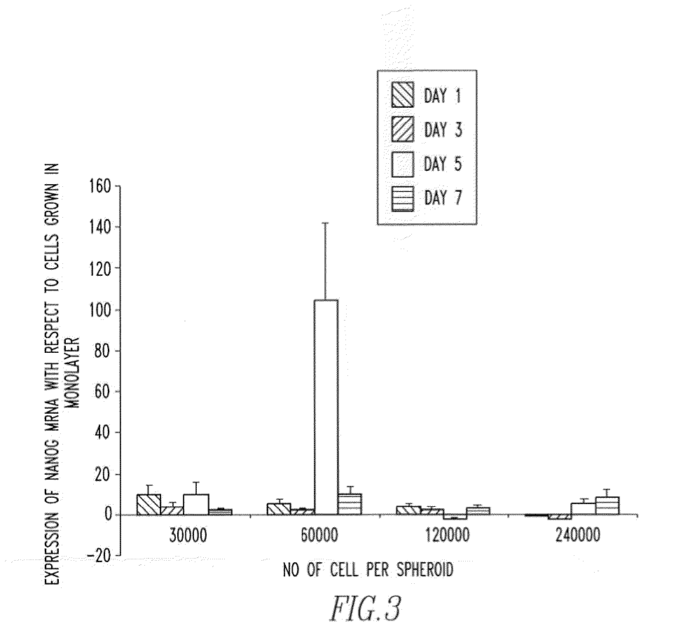

FIG. 2 represents the Oct-4 expression profile for spheroids of increasing size and increasing cell culture time. Oct-4 expression appears up-regulated in all spheroid culture conditions

ii) Nanog mRNA Expression in Human MSC Cell Spheroids

FIG. 3 represents the Nanog expression profile for spheroids of increasing size and increasing cell culture time. Nanog expression is relatively low in all culture conditions investigated. The clear exception to this is the Nanog expression from a 6×10⁴ cell seeded spheroid cultured for 5 days. A 100-fold increase in expression was observed under these conditions. This increase in expression is not apparent in any other seeding density. The expression is also transiently increased being low at days 3 and days 7. This suggests that at this cell seeding density and at this time, the cells are reverting to a less mature stem cell phenotype.

iii) Rex-1 mRNA Expression in Human MSC Cell Spheroids

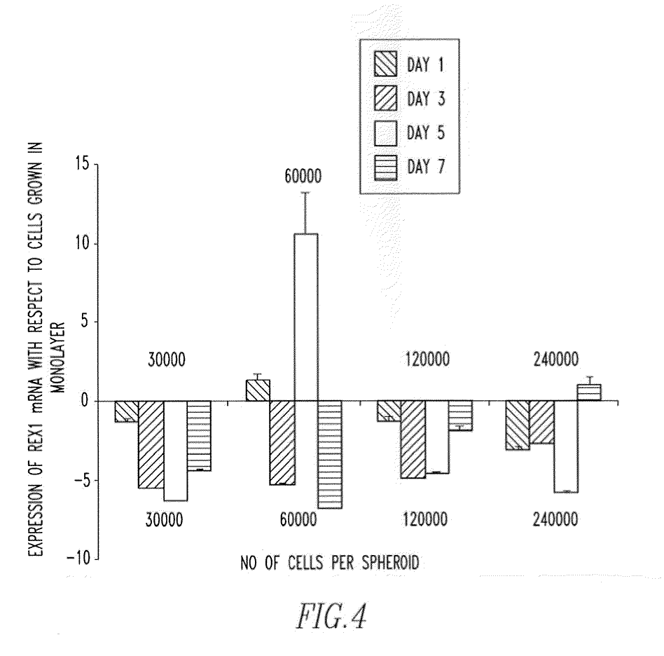

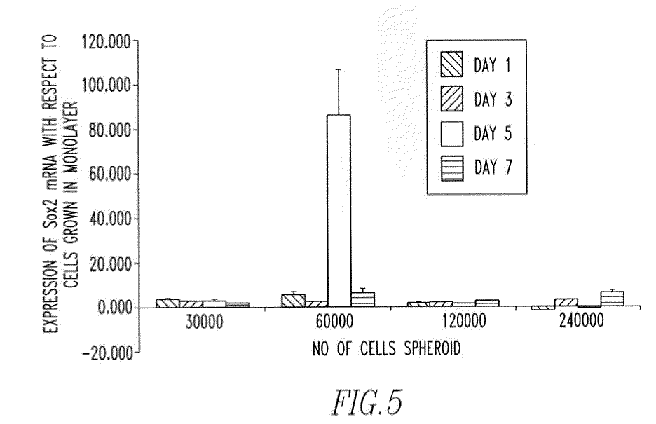

FIG. 4 represents the Rex-1 expression profile for spheroids of increasing size and increasing cell culture time. Rex-1 expression was low in all culture conditions, and often lower in expression than the cells in monolayer. However, under the same conditions that Nanog expression was transiently increased (see FIG. 3), Rex-1 expression is also higher. A 10-fold increase in expression was observed under these conditions. This increase in expression was not apparent in any other seeding density. These data, combined with the Nanog data suggest that the cells are reverting to a less mature stem cell phenotype.

iv) Sox-2 mRNA Expression in Human MSC Cell Spheroids

FIG. 5 represents the Sox-2 expression profile for spheroids of increasing size and increasing cell culture time. NSox-2 expression is relatively low in all culture conditions investigated. The clear exception to this is the Sox-2 expression from a 6×10⁴ cell seeded spheroid cultured for 5 days. A 80-fold increase in expression was observed under these conditions. This increase in expression is not apparent in any other seeding density. The expression is also transiently increased being low at days 3 and days 7. This suggests that at this cell seeding density and at this time, the cells are reverting to a less mature stem cell phenotype.

### Example 3

### Determination of the Expression of Embryonic Transcripts Following Culture of Human Dermal Fibroblasts in a 3D Environment

In order to determine whether or not the increase in embryonic transcripts were as a result of the 3D environment, the cell type or a combination of both, human dermal fibroblasts were cultured in 3D and the expression of Oct4, Nanog, SOX2 and Rex1 was determined.

### Materials and Methods

Method as in Example 1 and 2.

### Results

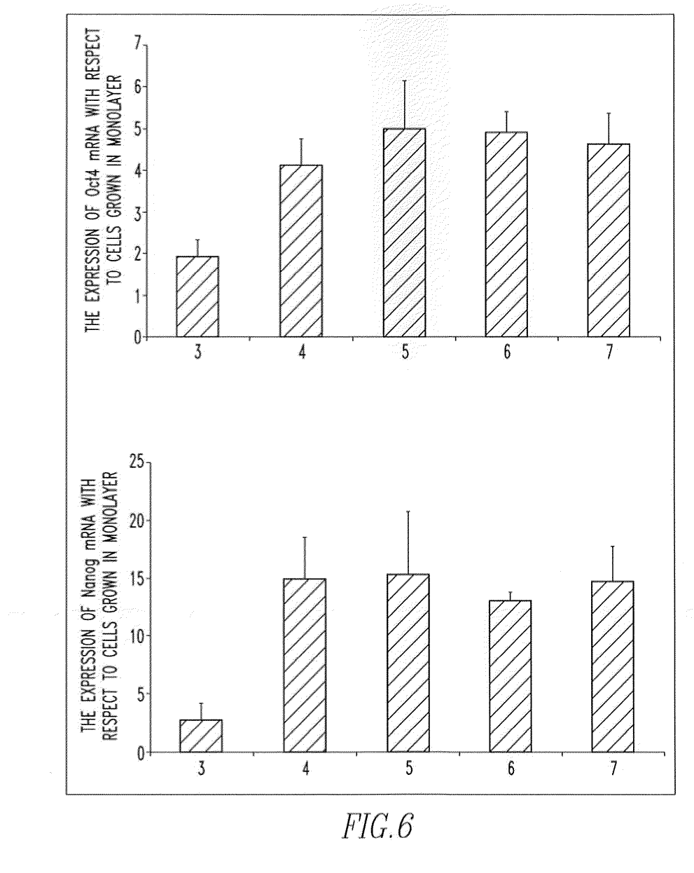

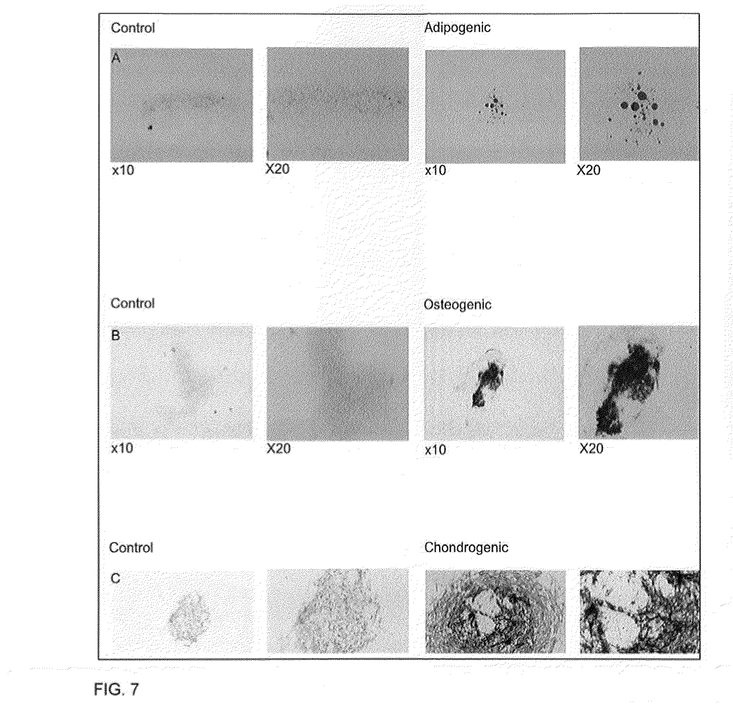

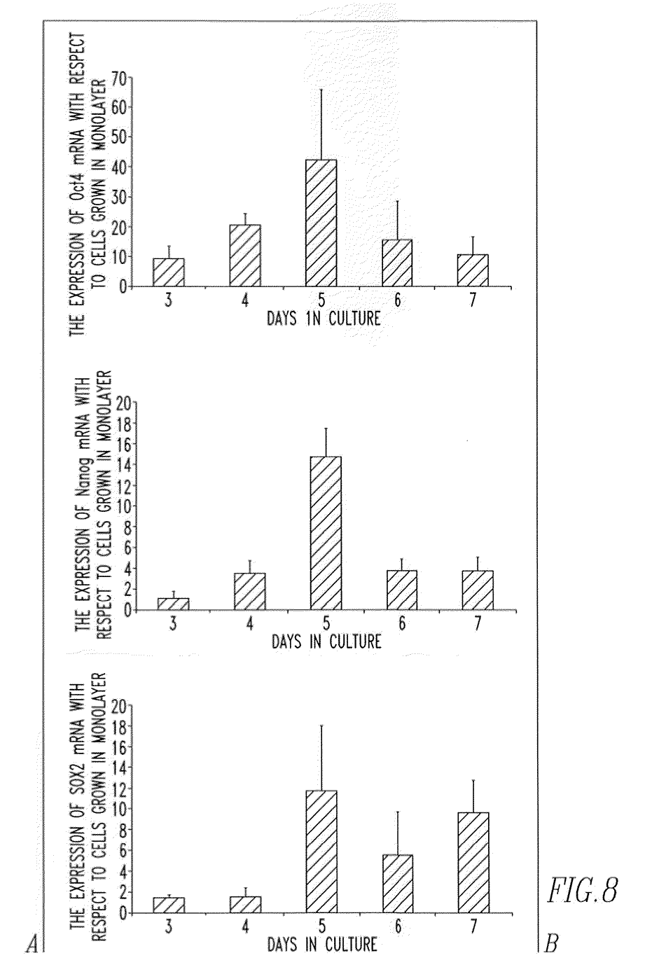

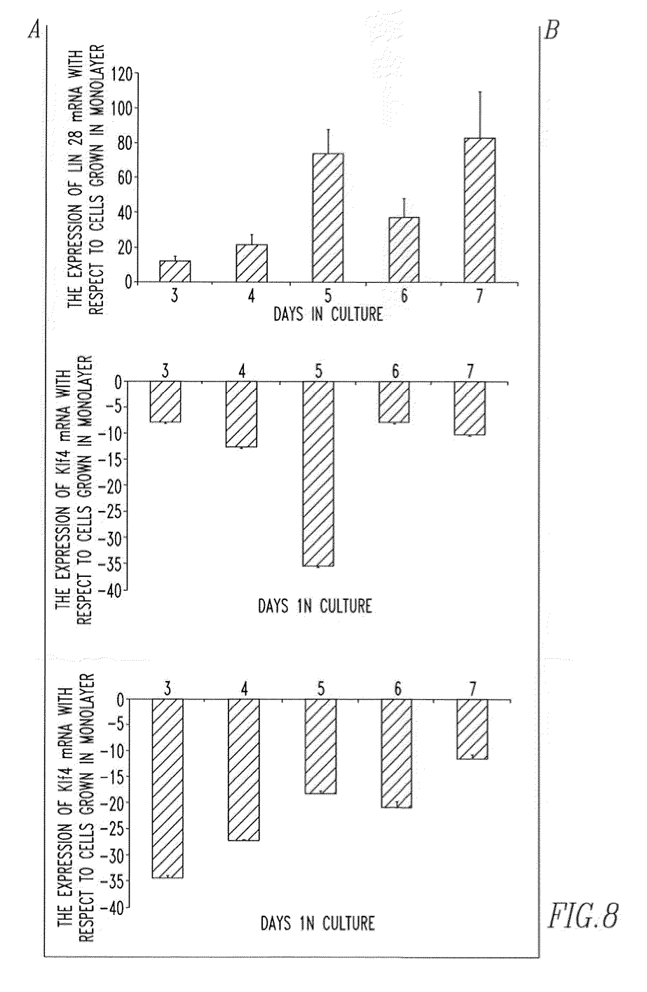

As FIG. 6 illustrates the expression of both Oct4 and Nanog were up-regulated in the 3D cultures, however the expression of Rex1 and SOX2 could not be detected. Such results suggest that the 3D culture does play an important role in the up-regulation of embryonic transcripts but that the effect is greater when more primitive cells (MSCs) are used.

### Example 4

### Establishment of the Potential of MSCs Cultured in 3D to Differentiate into Cell Types of the Mesoderm

It has been routinely reported in the literature that MSCs cultured in 2D can differentiate into cell types from the mesoderm germ layer. However their potential to differentiate into these cell types when cultured in 3D is unknown.

### Materials and Methods

Using routine protocols, MSC spheroids were differentiated down the osteogenic, chondrogenic and adipogenic lineages.

### Results

As FIG. 7 illustrates, MSCs cultured in 3D are able to differentiate down all three of the commonly reported lineages.

### Example 5

### Further Analysis of Embryonic Transcripts in the 3D Model

### Materials and Methods

Using the 3D model with 60,000 cells per spheroid, the expression of the transcripts C-Myc, KLF4 and Lin28 determined.

TABLE 2
Details of the primer sequences for
the qRT-PCR reactions

| Primer Name | Primer Sequence |
|---|---|
| Lin 28 Forward | CCCCCCAGTGGATGTCTT |
| Lin 28 Reverse | CCGGAACCCTTCCATGTG |
| C-Myc Forward | CGTCTCCACACATCAGCACAA |
| C-MYc Reverse | TCTTGGCAGCAGGATAGTCCTT |
| KLF4 Forward | CGCCACCCACACTTGTGAT |
| KLF4 Reverse | GTGCCTTGAGATGGGAACTCTT |

### Results

The expression of C-Myc and KLF4 were down regulated in the 3D model compared to the monolayer whereas the expression of the Lin28 transcript was up regulated.

As FIG. 8 shows, the four transcripts used to transduce the fibroblasts into iPS cells (Oct4, Nanog, Sox2 and Lin28) by Yu et al 2007 were all up-regulated in the 60,000 cell model at day 5.

### Example 6

### Establishment of Pluripotent Differentiation Capacity

Having established the multi-lineage differentiation potential of the MSCs when cultured in 3D, their potential to differentiate into cell types from other germ layers was subsequently investigated.

### [A] Cardiac Differentiation

### Materials and Methods

Spheroids were cultured in DMEM, 15% FBS, 1% P/S and 0.25% methyl cellulose. At days 3, 4, 5, 6 and 7 cells were induced to differentiate. The medium was replaced with RPMI-B27 supplemented with 100 ng/ml human recombinant Activin A for 24 hours—followed by 10 ng/ml human recombinant BMP4 for 4 days. The medium was then exchanged for RPMI-827 without supplementary cytokines—the cultures were re-fed every other day. At day 12 after the start of differentiation RNA was taken and qRT-PCR was carried out to determine the expression of cardiac markers Troponin, Myosin Light Chain (MLC), Myosin Heavy Chain (MHC) and MEF2c.

TABLE 3
Details of the primer sequences for
the qRT-PCR reactions

| Primer Name | Primer Sequerice |
|---|---|
| Troponin Forward | GGTCGTTCATGCCCAACTTG |
| Troponin Reverse | CCGGTGGATGTCATCAAAGTC |
| Myosin Light Chain | GCAAGGGCCCCATCAAC |
| Forward | |
| Myosin Light Chain | GGTCTGTCCCATTGAGCTTCTC |
| Reverse | |
| Myosin Heavy Chain | CCACCCAAGTTCGACAAAATC |
| Forward | |
| Myosin Heavy Chain | CGTAGCGATCCTTGAGGTTGTA |
| Reverse | |
| MEF2C Forward | TGAGAAAGAAGGGCCTTAATGG |
| MEF2C Reverse | CAGGGCTGTGACCTACGGAAT |

**** Expression was relative to monolayers which had also been differentiated down the cardiac lineage

### Results

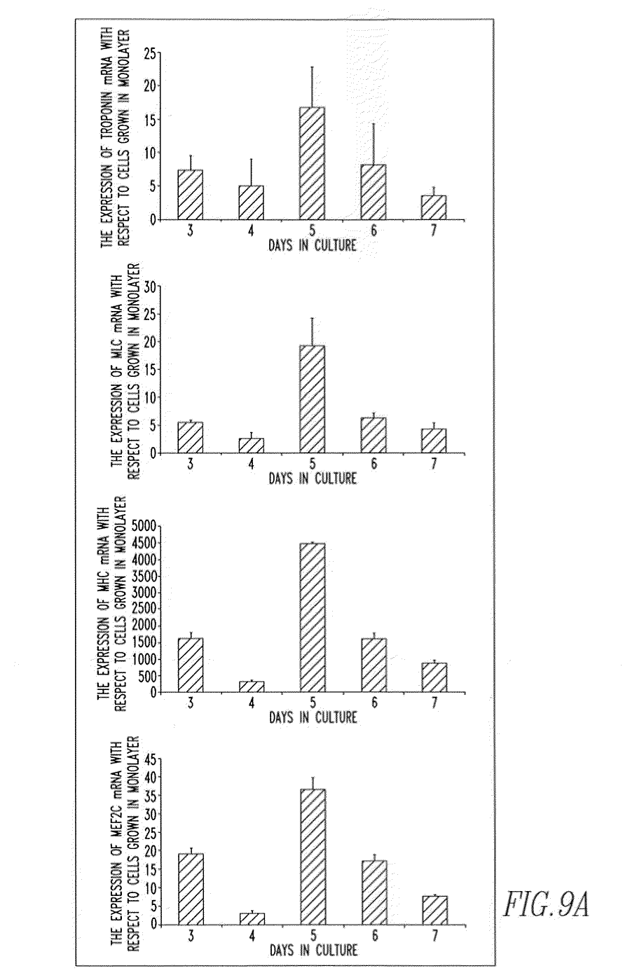

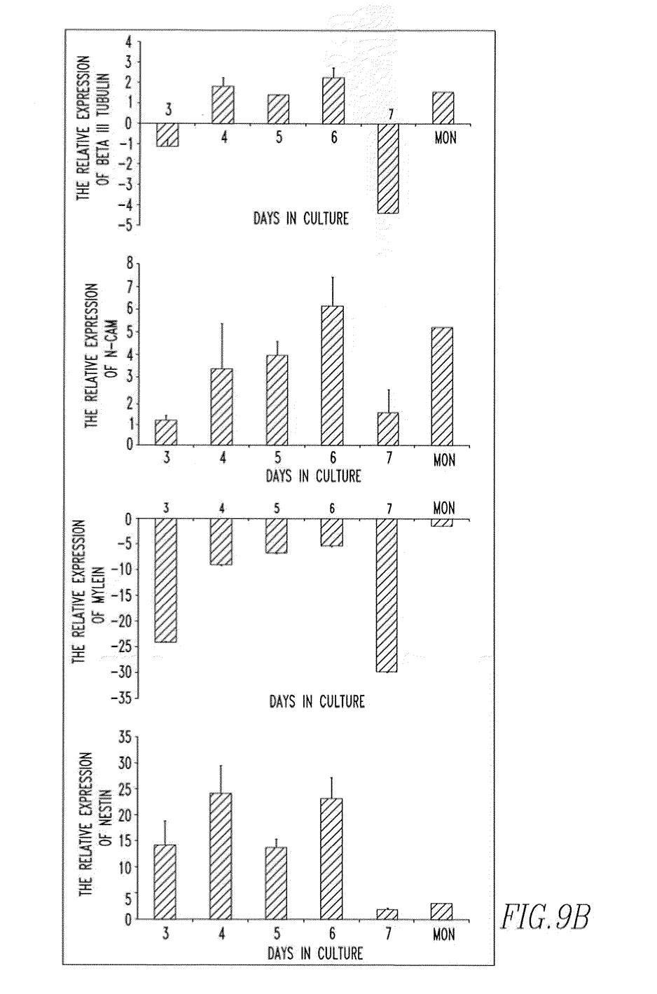

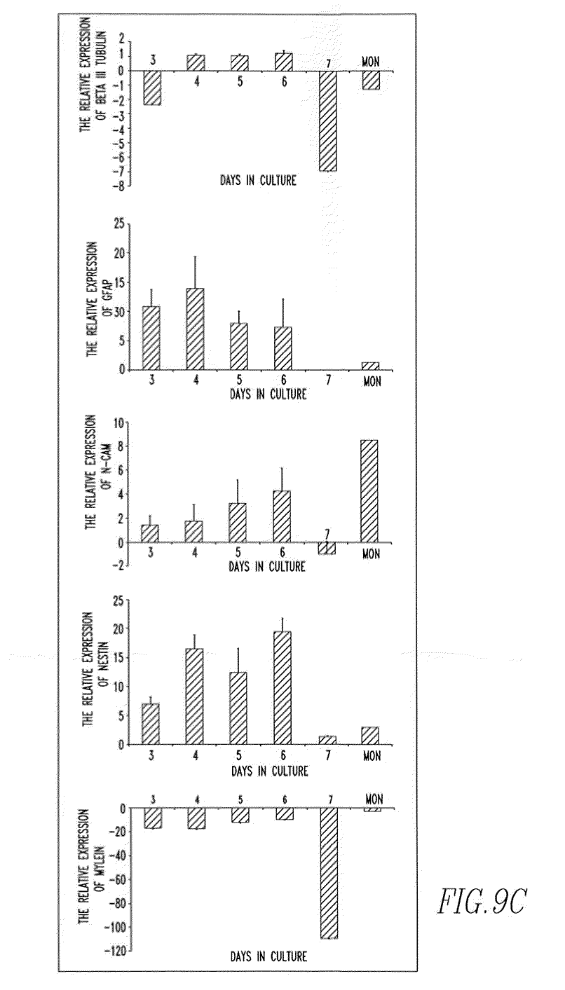

In comparison to the MSCs cultured in 2D the expression of all four transcripts were up regulated in the 3D model (see FIG. 9A). Interestingly the greatest up-regulation was seen from the spheroids which where induced down the cardiogenic lineage at day five of culture, where the expression of the embryonic transcripts Oct4, Nanog, SOX2 and Lin28 where at their highest.

### [B] Neuronal Differentiation

### Materials and Methods

Spheroids were cultured in DMEM, 15% FBS and 0.25% methyl cellulose. At days 3, 4, 5, 6 and 7 cells were induced to differentiate.

Stage 1: The medium was replaced with DMEM, 15% FBS, 1% P/S and 10 ng/ml βFGF for 24 hours.

Stage 2: The medium was replaced with DMEM, 15% FBS, 1% P/S and 1 mM β-mercaptoethanol and 10 ng/ml NT-3 for 48 hours.

Stage 3: The medium was replaced DMEM, 15% FBS, 1% P/S and (10 ng/ml NT-3), (10 ng/ml NGF), (50 ng/ml BDNF) for seven days.

RNA was taken after each stage and qRT-PCR carried out.

TABLE 4
Details of the primer sequences for
the qRT-PCR reactions

| Primer Name | Primer Sequence |
|---|---|
| Beta III Tubulin Forward | CAAGTTCTGGGAAGTCATCAGTGA |
| Beta III Tubulin Reverse | CCGAGTCGCCCACGTAGTT |
| Nestin Forward | TCCTGCTGTAGATGCAGAGATCAG |
| Nestin Reverse | AGGACGCTGGCAGGAATG |
| GFAP Forward | GAGATCCGCACGCAGTATGA |
| GFAP Reverse | ACTTGGAGCGGTACCACTCTTC |
| Mylein Forward | TTCCTCCCAAGGCACAGAGA |
| Mylein Reverse | CCCTGTCACCGCCAAAGA |
| N-CAM Forward | TCAGTGGTGTGGAATGATGATTC |
| N-CAM Reverse | CCGGCGTCGTCGATGT |

### Results

Over the time course of the three separate neuronal differentiation stages the 3D cultures exhibited an increase in the expression of the neuronal markers Nestin, N-CAM, GFAP and Beta III tubulin compared to the expression found in the undifferentiated monolayer cultures. A down regulation in the glial marker Myelin was also seen, suggesting that the 3D cultures were selectively differentiating down the neuronal lineage.

### Example 7

### Proliferation of Cells within the 3D Model

### Materials and Methods

The proliferation status of the optimised 3D model was determined. The expression of Ki67 a proliferation marker was determined using immunofluorescence for cells within the spheroid at days 3, 4, 5, 6 and 7 and compared to cells cultured in monolayer.

Cells were seeded on coverslips and left to adhere overnight. Spheroids were embedded in O.C.T and sectioned on the cryostat. All slides were fixed in 4% paraformaldehyde for 10 minutes. Slides and cover slips were then washed twice with PBS. Both were then blocked in 10% serum in 0.1 PBS-Tween (PBS-T) (goat serum) for 30 minutes at room temperature. The primary antibody (Rabbit polyclonal) Ki67 was then added (1/250) in 1% BSA PBS-T and left overnight at 4° C. The antibody was then removed and the slides washed three times with PBS. The secondary (goat anti-rabbit FITC) was then added (1/200 in 1% BSA-PBS-T) and left for 45 minutes at 4° C. in the dark. Finally the slides were washed three times with PBS 1% and mounted in Vectashield with PI or DAPI, and analysed using confocal microscopy

### Results

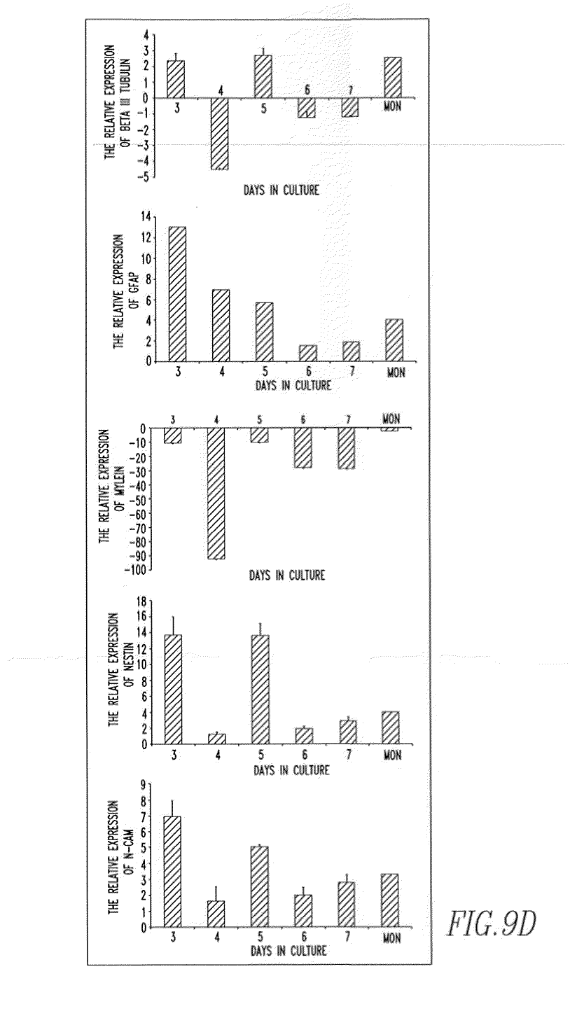

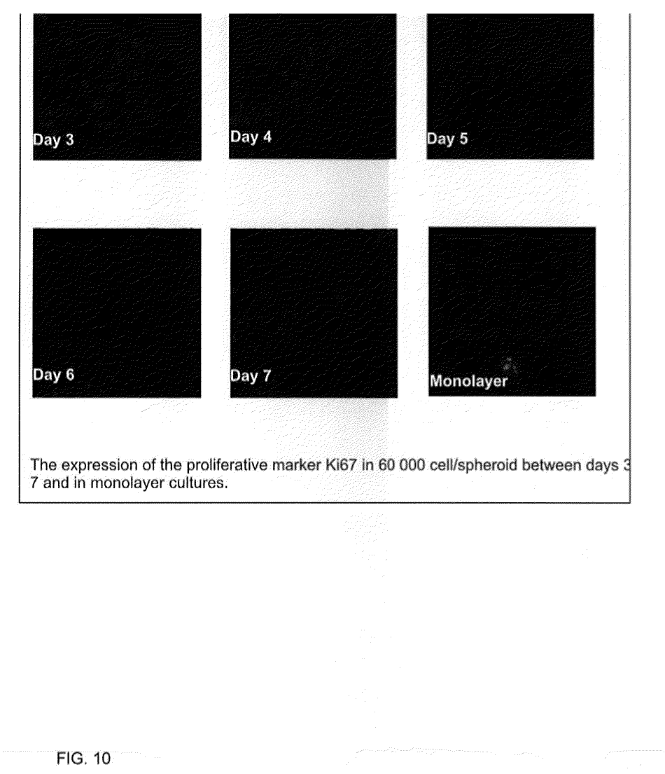

Positive staining could only be found in the monolayer samples, indicating that the cells within the 3D model were either quiescent or senescent (FIG. 10).

### Example 8

### Are the Cells in the 3D Model Senescent or Quiescent?

### Materials and Methods

MSCs which had previously been cultured in 3D were reintroduced back into 2D culture and the expression of the proliferation marker Ki67 investigated in order to determine if the non-proliferating cells are quiescent or senescent.

### Results

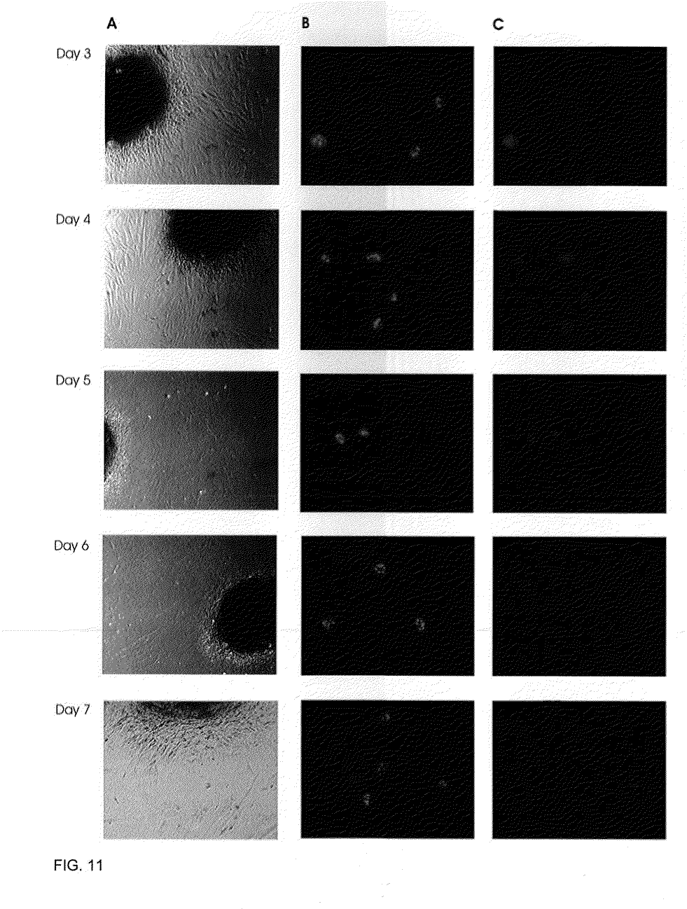

As FIG. 11 illustrates positive staining could be found in all samples, indicating that the MSCs were quiescent and not senescent as although the cells did not proliferate in 3D they were still able to when reintroduced back into a 2D environment. Bright field image (A) Immunofluorescence staining for the proliferation marker Ki67 (B) and DAPI staining (C).

### Example 9

### Organisation and Morphology of the MSCs in 3D Culture

### Materials and Methods

SEM images were taken of the cellular model from day 3-7.

### Results

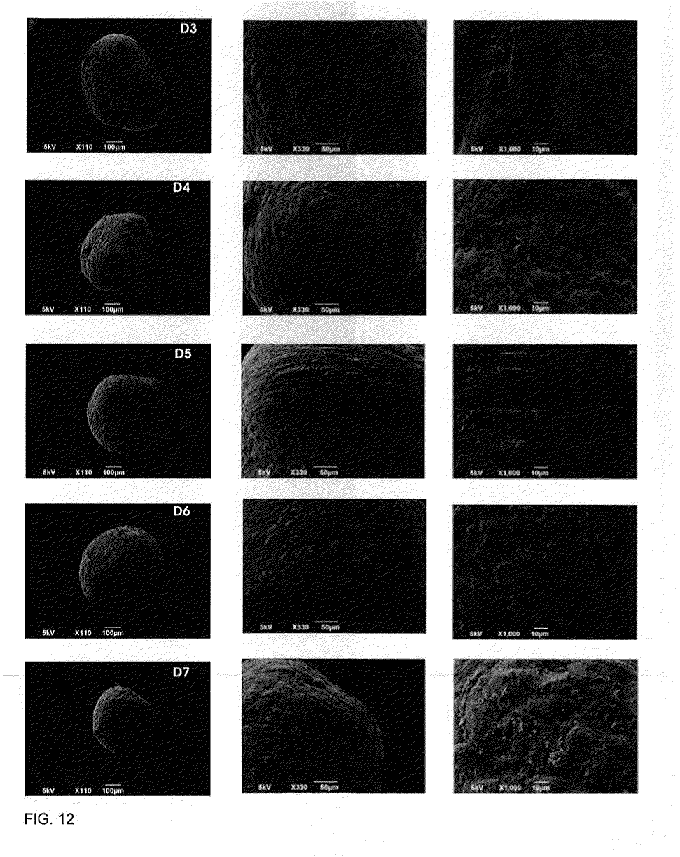

SEM images revealed compact spherical structures, with an initial smooth surface which became coarser as their time in culture progressed (see FIG. 12)

### Example 10

### Oxygen Consumption by the MSCs in the 3D Model

### Materials and Methods

The oxygen consumption of the cells in the MSC cellular 3D model was compared to the oxygen consumed by MSCs cultured in 2D.

Over a period of 90 minutes the oxygen consumption of spheroids which had been cultured between 1 and 7 days and cells which had been cultured in 2D culture was measured using a BD oxygen biosensor plate. To equilibrate the plate the oxygen biosensor plate was placed in the incubator overnight at 37° C. The next day 25 μl of pre-warmed Na₂SO₄ (100 mM) was placed in the first three wells of the plate to act as a positive control (0% oxygen). The plate was then placed in a fluorescence plate reader (set to 37° C.) and allowed to equilibrate for 30 minutes. The wells were then read and the gain set (Maximum fluorescence). To serve as 20% oxygen controls, 25 μl of pre warmed medium was added to separate wells in triplicate and sample wells were filled with 20 μl of medium and serial readings were taken every 2 minutes for 18 minutes. 5 μl of medium containing a spheroid or 60 000 cells cultured in 2D was added to each sample well. The plate was then sealed using a PCR foil seal and serial readings taken every 2 minutes for 90 minutes.

### Calculation

Fluorescence units were normalised to the blank values taken without the addition of any sample at all by dividing each well by its starting value (pre equilibrated but without medium). Each value was then normalised to the average value of the 20% medium control samples at each time point. These values were then transformed into μM oxygen using the following equation:

[O₂]=(DR/NRF−1)/Kₛᵥ

### Results

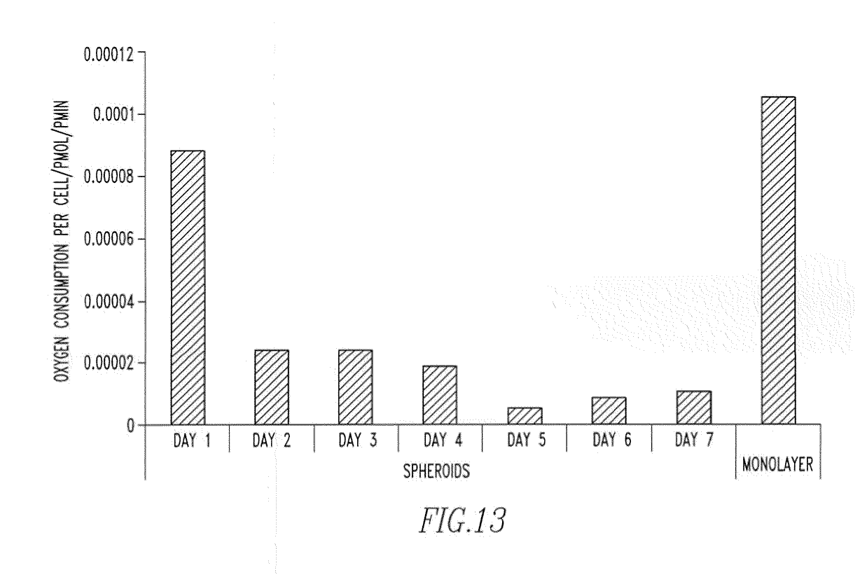

Having previously determined the oxygen consumption of an osteoblastic cell line in 3D culture, the oxygen consumption of the MSC cellular 3D model was compared to the oxygen consumed by MSCs cultured in 2D. Spheroids cultured for 24 hours appeared to consume a similar amount of oxygen to those cultured in 2D, with a significant decrease in oxygen consumed in spheroids cultured for a period of 48 hours and longer (see FIG. 13). These results indicate that the cells within the 3D model become less metabolically active after 48 hours.

### Example 11

### In vivo Study

### Showing Safety

### Materials and Methods

3 Groups of six mice each

### Group One

| | | |
|---|---|---|
| Monolayer MSCs | Donor FH337 x3 | x2 from each group |
| | | injected with PBS |
| | Donor FH181 x3 | x1 from each group |
| | | injected with Matrigel |

### Group Two

| | | |
|---|---|---|
| 3D MSCs | Donor FH337 x3 | x2 from each group |
| | | injected with PBS |
| | Donor FH181 x3 | x1 from each group |
| | | injected with Matrigel |

### Group Three

| | |
|---|---|
| Mouse E14 embryonic cells x3 | x2 from each group |
| | injected with PBS |
| | x1 from each group |
| | injected with Matrigel |
| Embryonic carcinoma cells | x2 from each group |
| (TERA2.cl.SP12) | injected with PBS |
| | x1 from each group |
| | injected with Matrigel |

Matrigel 9.8 mg/ml diluted 1:2

### Results

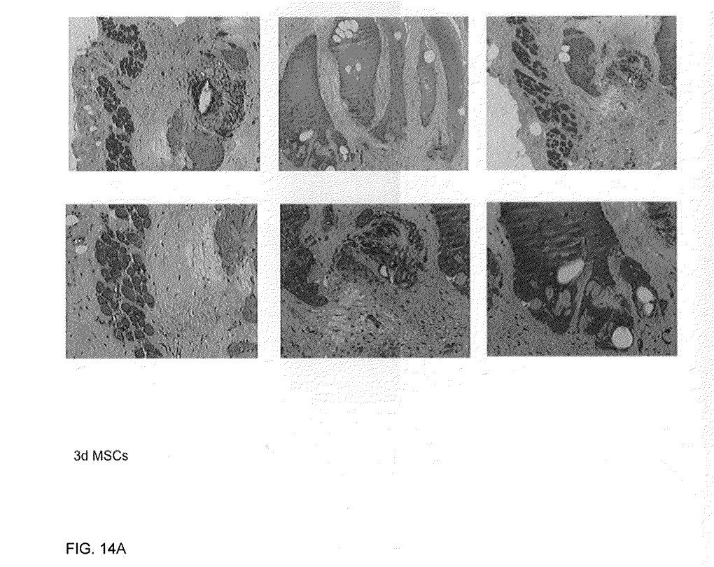

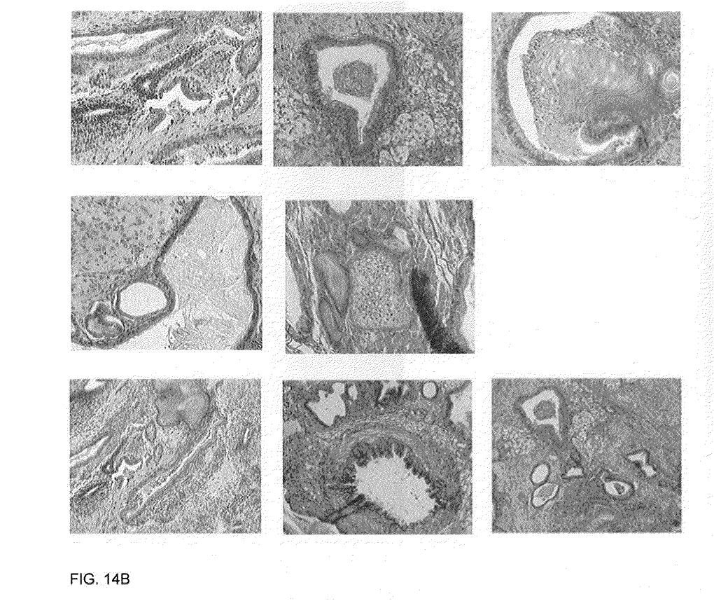

Tissue was only retrieved from one mouse injected with MSCs. This mouse was injected with 3D MSCs injected with Matrigel. The tissue mass was small in nature and from initial staining (H&E) it appears to consist of muscle and fat (FIG. 14A).

Mouse embryonic stem cells injected as positive controls formed large masses. On initial staining (H&E) the tissue seemed to contain structures from each of the three germ layers (FIG. 14B).

1. An in vitro method of culturing of non-embryonic cells such that the cells revert to a cell type expressing at least one gene expressed by an embryonic cell or embryonic-like, the method comprises the steps of;
i) providing non-embryonic cells,
ii) culturing the cells as a cellular aggregate for a period of between about 4 and 8 days at an initial cell density per aggregate of between about 3×10⁴ and 12×10⁴ cells.

2. A method according to claim 1, wherein the initial cell density of the aggregate is between about 5×10⁴ and 7×10⁴.

3. A method according to claim 1, wherein the initial cell density of the aggregate is about 6×10⁴.

4. A method according to claim 1, wherein the initial cell density of the aggregate is about 5×10⁴ and 7×10⁴ and the cells are cultured for about 5 days.

5. A method according to claim 1, wherein the initial cell density of the aggregate is about 6×10⁴ and the cells are cultured for about 5 days.

6. A method according to claim 5, wherein the non-embryonic cells are derived from fetal, neonatal or adult tissue.

7. A method according to claim 1, wherein the non-embryonic cells are non-embryonic stem cells.

8. A method according to claim 7, wherein the non-embryonic cell is a mesenchymal stem cell.

9. A method according to claim 7 wherein the non-embryonic cell is a haematopoietic stem cell.

10. A method according to claim 1, wherein the non-embryonic cell is a progenitor cell.

11. A method according to claim 1, wherein the non-embryonic cell is a differentiated cell.

12. A method according to claim 1, wherein the at least one gene expressed by an embryonic or embryonic-like cell is selected from the group consisting of Oct-4, Nanog, Rex-1, Sox-2 or telomerase, c-myc, KLF-4 or Lin28.

13. A cell aggregate comprising non-embryonic cells which have reverted to a cell type expressing at least one gene expressed by an embryonic cell or embryonic-like cell obtainable by culture of the non-embryonic cells according to claim 1.

14. A cell derived from a cell aggregate according to claim 13.

15. The use of a cell aggregate and/or a cell according to claim 13 for therapeutic purposes.

16. A composition comprising a cell aggregate and/or a cell according to claim 13 for use as a medicament.

17. The use of a cell aggregate and/or a cell according to claim 13, in the manufacture of a medicament for the therapeutic treatment of a connective tissue disorder.
